(19) United States
(12) Patent Application Publication
(10) Pub. No.: US 2025/0025281 A1
(43) Pub. Date: Jan. 23, 2025

(54) STENT WITH ANTI-OCCLUSION SYSTEM

(71) Applicant: BOSTON SCIENTIFIC SCIMED, INC., Maple Grove, MN (US)

(72) Inventors: Martyn G. Folan, Loughrea (IE); Louis McNern, Donegal (IE); Daniel Tuck, Galway (IE)

(73) Assignee: BOSTON SCIENTIFIC SCIMED, INC., Maple Grove, MN (US)

(21) Appl. No.: 18/774,348

(22) Filed: Jul. 16, 2024

Related U.S. Application Data
(60) Provisional application No. 63/514,024, filed on Jul. 17, 2023.

(51) Int. Cl. A61F 2/04

(52) U.S. Cl. CPC A61F 2/04; A61F 2002/041; A61F 2210/0076; A61F 2210/009; A61F 2230/0069; A61F 2240/001; A61F 2250/0001

(57) ABSTRACT

An illustrative stent includes an elongated tubular member comprising at least one strut forming a tubular wall having a plurality of cells extending through a thickness of the tubular wall. The elongated tubular member may be configured to move between a radially collapsed configuration and a radially expanded configuration. A coating may be disposed on the elongated tubular member and spanning at least some of the plurality of cells. One or more magnetic components may be disposed on or within the coating.

### CROSS REFERENCE TO RELATED APPLICATIONS

The present application claims the benefit of and priority to U.S. Provisional Patent Application Ser. No. 63/514,024, filed on Jul. 17, 2023, the disclosure of which is incorporated herein by reference.

### TECHNICAL FIELD

The present disclosure pertains to medical devices, methods for manufacturing medical devices, and uses thereof. More particularly, the present disclosure pertains to an anti-occlusion stent for implantation in a body lumen, and associated methods.

### BACKGROUND

Implantable stents are devices that are placed in a body lumen, such as the esophageal tract, the gastrointestinal tract (including the intestine, stomach and the colon), tracheobronchial tract, urinary tract, biliary tract, vascular system, etc. to provide support and to maintain the body lumen open. These stents are manufactured by any one of a variety of different manufacturing methods and may be used according to any one of a variety of methods. Of the known stents, delivery systems, and methods, each has certain advantages and disadvantages. For example, some stents may become occluded. Occlusive events can be a combination of local factors, such as, but not limited to inadequate stricture resolution and irregular bile properties and/or related to stent design, such as, but not limited to, coating tackiness and/or an interaction between the wire and bile. Thus, there is an ongoing need to provide alternative stent designs which offer opportunities for prophylactic intervention to delay occlusive events or potential occlusive sources without repeated intervention increasing stent efficacy during the procedural timespan.

### BRIEF SUMMARY

This disclosure provides design, material, manufacturing method, and use alternatives for medical devices. An example medical device may include a stent.

In one example, a stent may comprise an elongated tubular member comprising at least one strut forming a tubular wall having a plurality of cells extending through a thickness of the tubular wall, the elongated tubular member configured to move between a radially collapsed configuration and a radially expanded configuration, a coating disposed on the elongated tubular member and spanning at least some of the plurality of cells, and one or more magnetic components disposed on or within the coating.

Alternatively or additionally to any of the examples above, in another example, the coating may comprise an inner layer and an outer layer.

Alternatively or additionally to any of the examples above, in another example, the one or more magnetic components may be disposed between the inner layer and the outer layer of the coating.

Alternatively or additionally to any of the examples above, in another example, the one or more magnetic components may be discrete elements disposed within at least some of the plurality of cells.

Alternatively or additionally to any of the examples above, in another example, the one or more magnetic components may be elongate strips extending over the at least one strut.

Alternatively or additionally to any of the examples above, in another example, the one or more magnetic components may be spaced along a length of the elongated tubular member.

Alternatively or additionally to any of the examples above, in another example, the one or more magnetic components may be spaced about a circumference of the elongated tubular member.

Alternatively or additionally to any of the examples above, in another example, the coating may form a pocket within at least some of the plurality of cells.

Alternatively or additionally to any of the examples above, in another example, the coating may extend from a proximal end to a distal end of the elongated tubular member.

Alternatively or additionally to any of the examples above, in another example, the coating may cover less than an entirety of the elongated tubular member.

Alternatively or additionally to any of the examples above, in another example, at least one of the one or more magnetic components may be positioned adjacent to a coating-free region of the elongated tubular member.

Alternatively or additionally to any of the examples above, in another example, the one or more magnetic components may comprise a silicone base and a magnetic material.

Alternatively or additionally to any of the examples above, in another example, the magnetic material may comprise carbonyl iron.

Alternatively or additionally to any of the examples above, in another example, in response to an applied magnetic field, the one or more magnetic components may be configured to move radially inward and/or radially outward.

Alternatively or additionally to any of the examples above, in another example, the applied magnetic field may be a pulsed magnetic field or an alternating magnetic field.

In another example, a stent may comprise an elongated tubular member comprising at least one strut forming a tubular wall having a plurality of cells extending through a thickness of the tubular wall, the elongated tubular member configured to move between a radially collapsed configuration and a radially expanded configuration, a coating disposed on the elongated tubular member and spanning at least some of the plurality of cells, and a plurality of magnetic components embedded within the coating, the plurality of magnetic components including a silicone base and carbonyl iron. In response to an applied magnetic field, the one or more magnetic components may be configured to move radially inward and/or radially outward relative to the at least one strut.

In another example, a method for preventing occlusion of a stent may comprise delivering a stent to a target location within a body. The stent may comprise an elongated tubular member comprising at least one strut forming a tubular wall having a plurality of cells extending through a thickness of the tubular wall, the elongated tubular member configured to move between a radially collapsed configuration and a radially expanded configuration, a coating disposed on the elongated tubular member and spanning at least some of the plurality of cells, and one or more magnetic components disposed on or within the coating. The method may further comprise positioning a console configured to supply a magnetic field exterior to the body and adjacent to the target location and periodically activating the magnetic field. In response to the magnetic field being applied, the one or more magnetic components may be configured to move radially inward and/or radially outward relative to the at least one strut.

Alternatively or additionally to any of the examples above, in another example, the magnetic field may be a pulsed magnetic field.

Alternatively or additionally to any of the examples above, in another example, the magnetic field may be an alternating magnetic field.

Alternatively or additionally to any of the examples above, in another example, the console may be configured to be releasably secured about the body.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a side view of an illustrative endoluminal implant or stent;

FIG. 2A is a partial perspective view of the illustrative stent of FIG. 1;

FIG. 2B is a partial side view of the illustrative stent of FIG. 1;

FIG. 3 is a schematic cross-sectional view of the illustrative stent, taken at line 3-3 of FIG. 2B;

FIGS. 4A-4H are side views of the illustrative stent having magnetic components arranged in different regular or irregular arrangements;

FIG. 5A is a partial side view of an illustrative stent including magnetic components prior to magnetization;

FIG. 5B is a partial side view of the illustrative stent of FIG. 5A including magnetic components after magnetization;

FIG. 6 is a schematic view of the illustrative stent deployed within the biliary tree;

FIG. 7 is a schematic view of the illustrative placement of a console for generating a magnetic field relative to the body of the patient;

FIG. 8 is a perspective view of an illustrative console system;

FIGS. 9A-9C are partial cross-sectional views of the stent as a magnetic field is selectively applied; and

FIG. 10 is a side view of another illustrative endoluminal implant or stent.

While the disclosure is amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the term “about” may be indicative as including numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The detailed description and the drawings, which are not necessarily to scale, depict illustrative embodiments and are not intended to limit the scope of the disclosure. The illustrative embodiments depicted are intended only as exemplary. Selected features of any illustrative embodiment may be incorporated into an additional embodiment unless clearly stated to the contrary.

In some instances, it may be desirable to provide an endoluminal implant, or stent, that can deliver luminal patency within the pancreaticobiliary tree of a patient. The relatively narrow biliary tract ducts consist of a series of bifurcations linking the liver, gallbladder, and pancreas via the papilla to the duodenal space for the transportation of bile and related enzymic substances for many metabolic functions but most commonly the body's ability to digest and absorb fats and vitamins D and K. However, blockages may occur in the pancreaticobiliary tree due to tumor related duct narrowing, stricture formation, infections, or stone and sludge generation among other etiologies. Endoscopic retrograde cholangiopancreatography (ERCP) is used to diagnose and treat these duct narrowings, regardless of the malignant or benign nature of the disease. Commonly a fully covered self-expanding metal stent (SEMS) may be used such that the radial forces of the stent scaffold the stricture. However, occlusions may occur within the stent after stent placement.

Occlusive events can be a combination of local factors such as, but not limited to, inadequate stricture resolution and/or irregular bile properties. Occlusion can also be related to stent design components such as, but not limited to, coating tackiness and/or an interaction between the wire of the stent and bile (both causing drag and pooling of bile which accumulates over a time course). Due to the remote nature of a placed SEMS, the ability to diagnose and interact with a potential occlusive event is limited and typically can only be resolved through intervention at a stage when the occlusive event has demonstrated an exacerbation of a patient's symptoms (e.g., jaundice, abdominal pain, etc.). Occlusion may result in increased procedural charges due to reintervention and stent replacement, patient discomfort even though the stent device itself may be functioning effectively as a scaffold, retarding of the vessel, and/or an unjustified reputation for particular device families as being less efficacious or brands spurned due to historical or anecdotal knowledge of occlusive or suggestive occlusive events. The present disclosure is directed towards alternative stent designs which offer opportunities for prophylactic or preventative intervention to delay occlusive events or potential occlusive sources without repeated surgical intervention increasing stent efficacy during the procedural timespan. While the present disclosure is described with respect to the pancreaticobiliary ductal system, the devices, systems, and/or methods described herein may be used in stents or endoluminal implants positioned in other parts of the body, such as, but not limited to, bodily tissue, bodily organs, vascular lumens, non-vascular lumens and combinations thereof, such as, but not limited to, in the coronary or peripheral vasculature, trachea, bronchi, colon, small intestine, esophagus, biliary tract, urinary tract, prostate, brain, stomach, and the like.

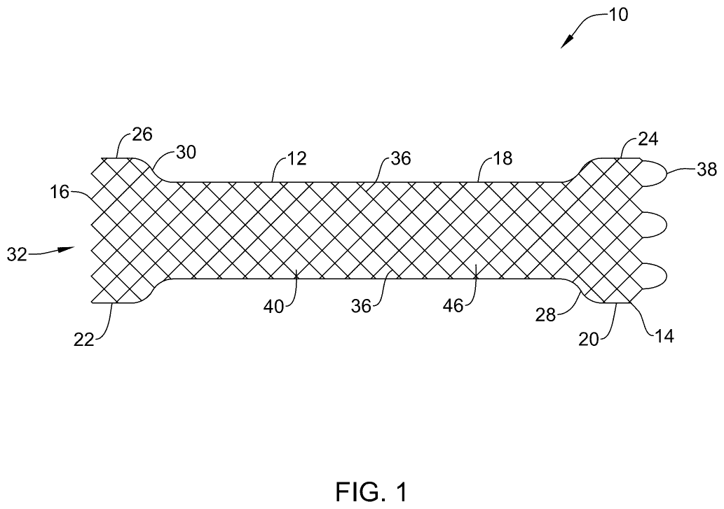

FIG. 1 illustrates a side view of an illustrative endoluminal implant 10, such as, but not limited to, a stent. In some instances, the stent 10 may be formed from an elongated tubular member 12. While the stent 10 is described as generally tubular, it is contemplated that the stent 10 may take any cross-sectional shape desired. The stent 10 may have a first, or proximal end 14, a second, or distal end 16, and an intermediate region 18 disposed between the first end 14 and the second end 16. The stent 10 may include a lumen 32 extending from a first opening adjacent the first end 14 to a second opening adjacent to the second end 16 to allow for the passage of food, fluids, etc.

The stent 10 may be expandable from a first radially collapsed configuration (not explicitly shown) to a second radially expanded configuration. In some cases, the stent 10 may be deployed to a configuration between the collapsed configuration and a fully expanded configuration. The stent 10 may be structured to extend across a stricture and to apply a radially outward pressure to the stricture in a lumen to open the lumen and allow for the passage of substances.

In some embodiments, the proximal end 14 of the stent 10 may include a plurality of loops 38. The loops 38 may be configured to receive a retrieval tether or suture (not explicitly shown) interwoven therethrough, or otherwise passing through one or more of the loops 38. The retrieval suture may be used to collapse and retrieve the stent 10, if so desired. For example, the retrieval suture may be pulled like a drawstring to radially collapse the proximal end 14 of the stent 10 to facilitate removal of the stent 10 from a body lumen.

The stent 10 may have a woven structure, fabricated from a number of filaments or struts 36 forming a tubular wall. In some embodiments, the stent 10 may be knitted or braided with a single filament or strut interwoven with itself and defining open cells 46 extending through the thickness of the tubular wall of the stent 10. In other embodiments, the stent 10 may be braided with several filaments or struts interwoven together and defining open cells 46 extending along a length and around the circumference of the tubular wall of the stent 10. The open cells 46 may each define an opening from an outer surface of the tubular wall to an inner surface of the tubular wall (e.g., through a thickness thereof) that is free from the filaments or struts 36. Some exemplary stents including braided filaments include the WallFlex®, WALLSTENT®, and Polyflex® stents, made and distributed by Boston Scientific, Corporation. In another embodiment, the stent 10 may be knitted, such as the Ultraflex™ stents made by Boston Scientific, Corporation. In yet another embodiment, the stent 10 may be of a knotted type, such the Precision Colonic™ stents made by Boston Scientific, Corporation. In still another embodiment, the stent 10 may be a laser cut tubular member, such as the EPIC™ stents made by Boston Scientific, Corporation. A laser cut tubular member may have an open and/or closed cell geometry including one or more interconnected monolithic filaments or struts defining open cells 46 therebetween, with the open cells 46 extending along a length and around the circumference of the tubular wall. The open cells 46 may each define an opening from an outer surface of the tubular wall to an inner surface of the tubular wall (e.g., through a thickness thereof) that is free from the interconnected monolithic filaments or struts. In some instances, an inner and/or outer surface of the tubular wall of the stent 10 may be entirely, substantially, or partially, covered with a polymeric covering or coating 40, as will be described in more detail herein. The covering or coating 40 may extend across and/or occlude one or more, or a plurality of the cells 46 defined by the struts or filaments 36. In some cases, the stent 10 may be a self-expanding stent (SES), although this is not required.

In some instances, in the radially expanded configuration, the stent 10 may include a first end region 20 proximate the proximal end 14 and a second end region 22 proximate the second end 16. In some embodiments, the first end region 20 and the second end region 22 may include retention features or anti-migration flared regions 24, 26 having enlarged diameters relative to the intermediate portion 18. The anti-migration flared regions 24, 26, which may be positioned adjacent to the first end 14 and the second end 16 of the stent 10, may be configured to engage an interior portion of the walls of the body lumen. In some embodiments, the retention features, or flared regions 24, 26 may have a larger diameter than the cylindrical intermediate region 18 of the stent 10 to prevent the stent 10 from migrating once placed in the body lumen. It is contemplated that the transition 28, 30 from the cross-sectional area of the intermediate region 18 to the retention features or flared regions 24, 26 may be gradual, sloped, or occur in an abrupt step-wise manner, as desired.

In some embodiments, the first anti-migration flared region 24 may have a first outer diameter and the second anti-migration flared region 26 may have a second outer diameter. In some instances, the first and second outer diameters may be approximately the same, while in other instances, the first and second outer diameters may be different. In some embodiments, the stent 10 may include only one or none of the anti-migration flared regions 24, 26. For example, the first end region 20 may include an anti-migration flare 24 while the second end region 22 may have an outer diameter similar to the intermediate region 18. It is further contemplated that the second end region 22 may include an anti-migration flare 26 while the first end region 20 may have an outer diameter similar to an outer diameter of the intermediate region 18. In some embodiments, the stent 10 may have a uniform outer diameter from the first end 14 to the second end 16. It is contemplated that the outer diameter of the stent 10 may be varied to suit the desired application.

It is contemplated that the elongated tubular member of the stent 10 can be made from a number of different materials such as, but not limited to, metals, metal alloys, shape memory alloys, and/or polymers, as desired, enabling the stent 10 to be expanded into shape when accurately positioned within the body. In some instances, the material may be selected to enable the stent 10 to be removed with relative ease as well. For example, the elongated tubular member of the stent 10 can be formed from alloys such as, but not limited to, nitinol and Elgiloy®. Depending on the material selected for construction, the stent 10 may be self-expanding or require an external force to expand the stent 10. In some embodiments, composite filaments may be used to make the stent 10, which may include, for example, an outer shell or cladding made of nitinol and a core formed of platinum or other radiopaque material. It is further contemplated the elongated tubular member of the stent 10 may be formed from polymers including, but not limited to, polyethylene terephthalate (PET). In some instances, the filaments of the stent 10, or portions thereof, may be bioabsorbable or biodegradable, while in other instances the filaments of the stent 10, or portions thereof, may be biostable.

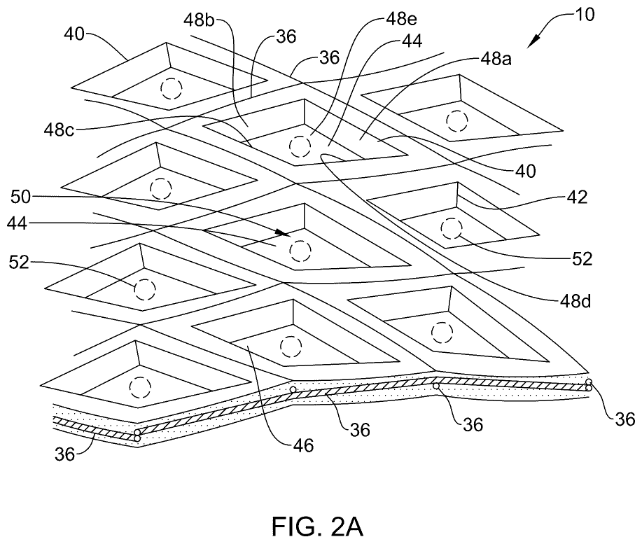

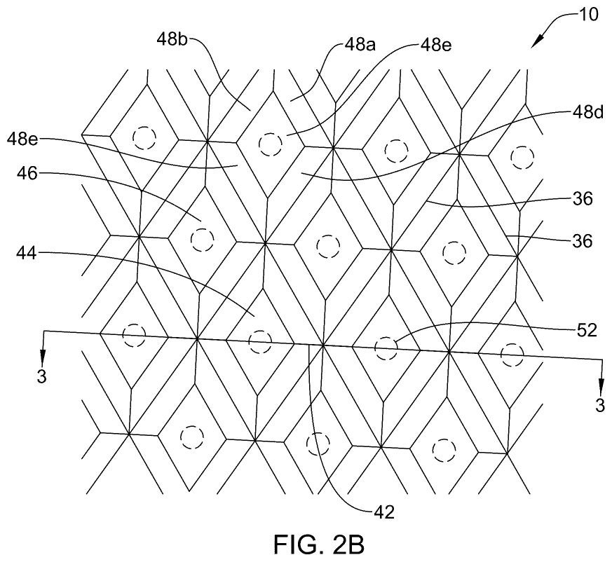

FIG. 2A illustrates a partial perspective view of the illustrative stent 10 of FIG. 1 and FIG. 2B illustrates a partial side view of the illustrative stent 10 of FIG. 1. As described above, the inner and/or outer surface of the tubular wall of the stent 10 may be entirely, substantially, or partially covered with a polymeric covering or coating 40. The coating 40 may be silicone, polyurethane, or other flexible polymeric material. The coating 40 may be applied such that there is an excess of material or a pocket of material 44 extending between the struts 36. For example, instead of extending generally taut in the same plane as the struts 36, the coating 40 may be loose and extend radially inward from the struts 36 for a radial distance or height 42 to form a void. However, in some cases, the coating 40 may be generally taut and extend in the same plane as the struts 36. While FIGS. 2A and 2B illustrate the pockets 44 as extending radially inward, it is contemplated that the pockets 44 may extend radially outward from the struts 36 for a radial distance or height. In some instances, the pocket 44 may have a generally truncated pyramidal shape with four flat converging (e.g., angled) side walls 48a, 48b, 48c, 48d and a flat bottom or base wall 48c. While the pocket 44 is generally illustrated as having a cross-section in the shape of a rhombus similar to a diamond shape of the cells 46 formed by the struts 36, the pocket 44 may take any shape desired. For instance, in some instances the pocket 44 may have a base wall 48e having a generally arcuate shape, such as a generally spherical shape with a spherically concave radially outward facing surface and a spherically convex radially inwardly facing surface. The pocket 44 may form a void 50 between a radially outward surface (e.g., a base surface) of the pocket 44 and the circumference of the tubular wall of the stent 10 formed by the struts 36. When the pockets 44 extends radially outward from the tubular wall of the stent 10, the void 50 may be radially outward of the tubular wall of the stent 10.

In some instances, the pockets 44 may be formed using a mandrel and/or mold. For example, a mandrel may be formed having protrusions or recesses of the desired size and shape of the pockets 44. The struts 36 may be wound, braided, woven, or otherwise disposed about the mandrel with the cells of the tubular wall aligned with the protrusions or recesses. A sleeve made of, for example, silicone or other polymer material, may be disposed over the mandrel and struts 36. The sleeve may be heated or otherwise molded to the shape of the mandrel to form the coated stent 10 including the pockets 44 between the struts 36. Alternatively, a polymeric material may be spray or dip coated onto the mandrel and struts 36 such that the polymeric material flows over the protrusions and/or into the recesses of the mandrel. It is contemplated that either radially inwardly extending pockets 44 or radially outwardly extending pockets 44 may be formed using either protrusions or recesses as the coating 40 may be flexible enough to allow the pockets 44 to be inverted to the desired configuration.

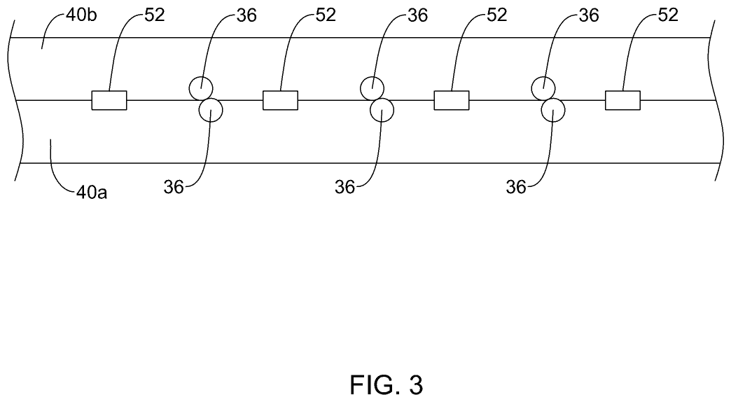

Referring additionally to FIG. 3 which illustrates a schematic cross-sectional view of the illustrative stent 10, taken at line 3-3 of FIG. 2B, the pockets 44 may further include a magnetic component 52 such that the stent 10 includes at least one or more magnetic components 52. In some instances, the magnetic components 52 may be secured to the coating, such as in the pockets 44, while the magnetic components 52 are spaced away from and/or not in direct contact with the struts 36 of the stent 10. In other words, in some instances the magnetic components 52 may be positioned which the cells 46 between adjacent filaments or struts 36 of the stent 10 while not directly contacting the filaments or struts 36 forming the scaffold structure of the stent 10. In some embodiments, the magnetic component 52 may be encapsulated between an inner layer 40a and an outer layer 40b of the coating 40. However, this is not required. In some cases, the magnetic component 52 may be on an inner surface of the inner layer 40a or an outer surface of the outer layer 40b of the coating 40. As described above, in some instances, an inner and/or outer surface of the scaffold structure of the stent 10 may be entirely, substantially or partially, covered with a polymeric covering or layer 40. For example, a covering or coating 40 may extend across the open cells 46 of the scaffold structure to prevent tissue ingrowth into the lumen of the stent 10. However, in some embodiments one or both of the polymeric coverings 40a, 40b may be omitted. For example, in some embodiments the stent 10 may include only the outer polymeric covering 40b on an outer surface of the scaffold structure. In other embodiments, the stent 10 may include only the inner polymeric covering 40a on an inner surface of the scaffold structure. In some instances, the inner layer 40a and the outer layer 40b may be formed as a single unitary structure. In other embodiments, the inner layer 40a and the outer layer 40b may be formed as separate layers. The inner and outer layers 40a, 40b may be formed from the same material or different materials, as desired. It is contemplated that the inner layer 40a and/or outer layer 40b may be applied such that the elasticity of the coating 40 allows for the localized movement of the magnetic components 52. The inner layer 40a and/or outer layer 40b may span or be disposed within openings or interstices 46 defined between adjacent stent filaments or struts 36 of the scaffold structure. It can be appreciated that as inner layer 40a and outer layer 40b extend outwardly and inwardly, respectively, they may touch and/or form an interface region within the spaces (e.g., openings, cells, interstices) 46 in the wall of the scaffold structure of the stent 10. For example, the detailed view of FIG. 3 shows that both the inner and outer layers 40a, 40b may extend into the openings 46 defined between adjacent stent struts 36 and form an interface region. Further, the inner and outer layers 40a, 40b may additionally extend between adjacent filaments or struts 36, thereby filling any space between adjacent filaments or strut members 36, and thus prevent tissue ingrowth into the lumen of the stent 10.

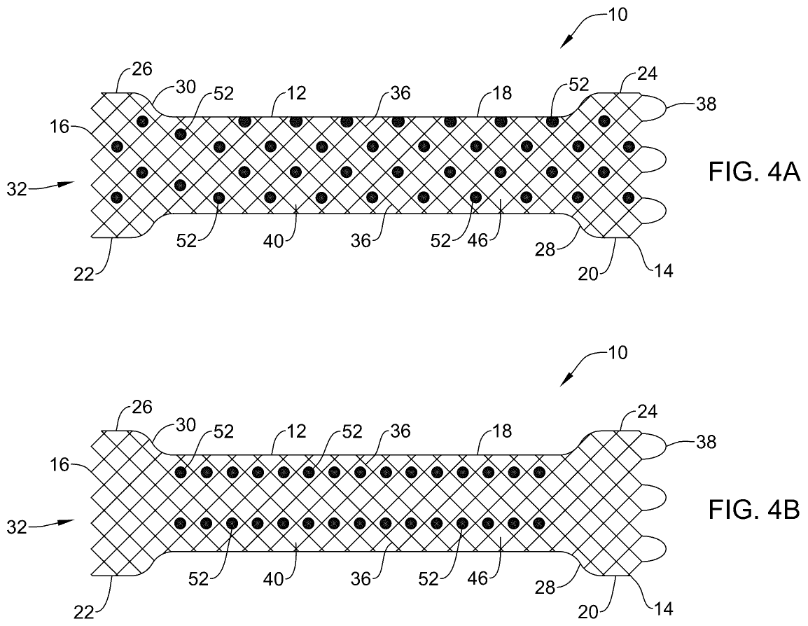

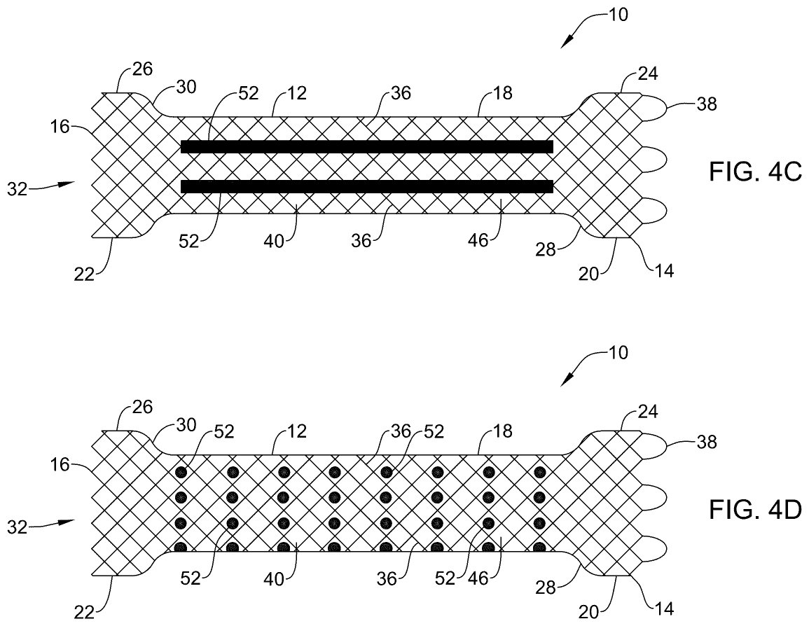

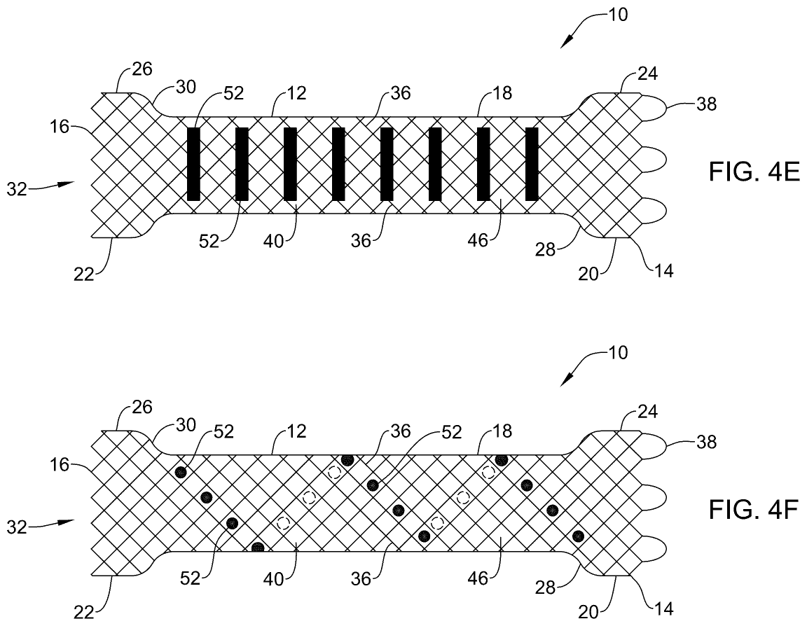

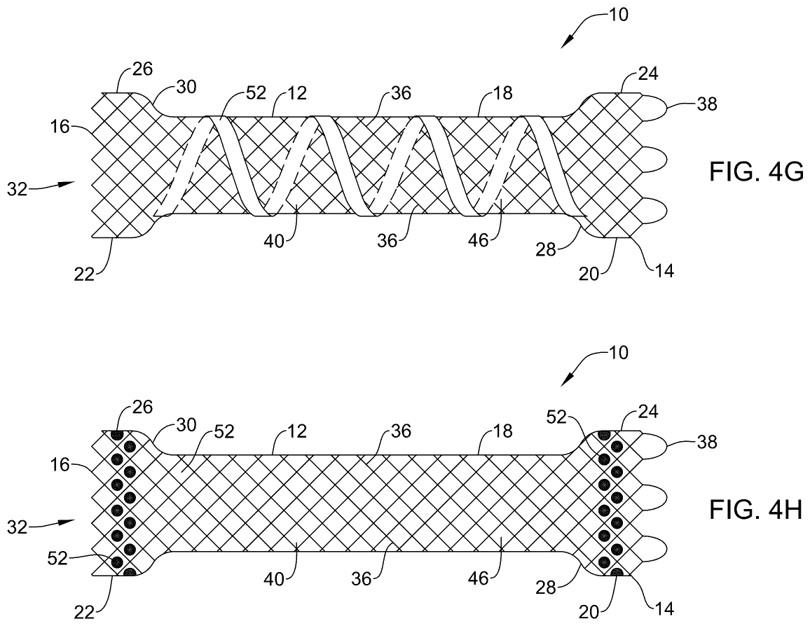

The magnetic components 52 may be arranged in a number of different patterns such that the magnetic components 52 extend over a length and/or circumference of the stent 10. In some cases, each opening 46 may include a magnetic component 52 as shown in FIGS. 2A and 2B. However, this is not required. FIGS. 4A-4H illustrate side views of the illustrative stent 10 having magnetic components 52 arranged in different regular or irregular arrangements. One or more magnetic components 52 may be spaced along a length and/or circumference of the stent 10. Some openings 46 may be free from or may not include a magnetic component 52, as shown FIG. 4A. While FIG. 4A illustrates the magnetic components 52 as generally uniformly distributed about a length of the stent 10, this is not required. In another example, the magnetic components 52 may be arranged in one or more longitudinal arrays, as shown in FIG. 4B. The longitudinal arrays may be evenly or eccentrically spaced about a circumference of the stent 10 and may be spaced about an entirety of the circumference or less than an entirety of the circumference of the stent 10. Further, the stent 10 may include only a single array or more than one array of magnetic components 52, as desired. The longitudinally extending arrays may extend along an entirety of a length of the stent 10 or less than an entirety of the length of the stent 10, as desired. The longitudinal arrays need not extend continuously along a length of the stent 10. For example, the arrays may include a gap or space therein.

Further, in some embodiments, the magnetic components 52 may not be confined to being positioned within the openings 46. For example, the magnetic components 52 may be positioned over and/or under one or more struts 36. In another example, the magnetic components 52 may be arranged as one or more longitudinally extending magnetic components 52, as shown in FIG. 4C. The longitudinally extending magnetic components 52 may be evenly or eccentrically spaced about a circumference of the stent 10 and may be spaced about an entirety of the circumference or less than an entirety of the circumference of the stent 10. Further, the stent 10 may include only a single longitudinally extending magnetic component 52 or more than one longitudinally extending magnetic components 52, as desired. The longitudinally extending magnetic components 52 may extend along an entirety of a length of the stent 10 or less than an entirety of the length of the stent 10, as desired. The longitudinally extending magnetic components 52 need not extend continuously along a length of the stent 10. For example, the longitudinally extending magnetic components 52 may include gaps or spaces therein.

In another example, the magnetic components 52 may be arranged in one or more circumferential arrays, as shown in FIG. 4D. The circumferential arrays may be evenly or eccentrically spaced about a length of the stent 10 and may be spaced about an entirety of the length or less than an entirety of the length of the stent 10. Further, the stent 10 may include only a single array or more than one array of magnetic components 52, as desired. The circumferentially extending arrays may extend about an entirety of a circumference of the stent 10 or less than an entirety of the circumference of the stent 10, as desired. The circumferentially extending arrays need not extend continuously along a circumference of the stent 10. For example, the circumferentially extending arrays may include gaps or spaces therein. In another example, the magnetic components 52 may be arranged as one or more circumferentially extending magnetic components 52, as shown in FIG. 4E. The circumferentially extending magnetic components 52 may be evenly or eccentrically spaced about a length of the stent 10 and may be spaced about an entirety of the length or less than an entirety of the length of the stent 10. Further, the stent 10 may include only a single circumferentially extending magnetic component 52 or more than one circumferentially extending magnetic components 52, as desired. The circumferentially extending magnetic components 52 may extend about an entirety of a circumference of the stent 10 or less than an entirety of the circumference of the stent 10, as desired. The circumferentially extending magnetic components 52 need not extend continuously along a circumference of the stent 10. For example, the circumferentially extending magnetic components 52 may include gaps or spaces therein.

In another example, the magnetic components 52 may be arranged in one or more helical arrays, as shown in FIG. 4F. The helical arrays may be evenly or eccentrically spaced about a length of the stent 10 and may be spaced or extend along an entirety of the length and/or circumference or less than an entirety of the length and/or circumference of the stent 10. Further, the stent 10 may include only a single array or more than one array of magnetic components 52, as desired. The helically extending arrays need not extend continuously along a length and/or circumference of the stent 10. For example, the helically extending arrays may include gaps or spaces therein. In another example, the magnetic components 52 may be arranged as one or more helically extending magnetic components 52, as shown in FIG. 4G. The helically extending magnetic component 52 may be evenly or eccentrically spaced about a length of the stent 10 and may be spaced or extend along an entirety of the length and/or circumference or less than an entirety of the length and/or circumference of the stent 10. Further, the stent 10 may include only a single helically extending magnetic component 52 or more than one helically extending magnetic components 52, as desired. The helically extending magnetic components 52 need not extend continuously along a length and/or circumference of the stent 10. For example, the helically extending magnetic components 52 may include gaps or spaces therein.

It is further contemplated that the magnetic components 52 may be clustered at the proximal end 14 of the stent 10 and/or at the distal end 16 of the stent 10 while the intermediate region 18 is free from magnetic components 52, as shown in FIG. 4H. In yet other examples, the intermediate region 18 may include magnetic components 52 while the proximal end 14 and/or distal end 16 of the stent 10 are free from magnetic components 52. These are just some examples of potential arrangements of the magnetic components 52. It should be understood that the magnetic components 52 may be arranged in any arrangement, regular or irregular, as desired. While FIGS. 4A-4H generally illustrate the magnetic components 52 as discrete elements within the openings 46 between struts 36 or as elongate strips, in some cases, the magnetic components 52 may be incorporated into an entirety of the coating 40 or selected regions thereof. It is further contemplated that the stent 10 may include the magnetic components 52 as any combination of discrete elements within the openings 46, elongate strips extending over at least one strut 36, and/or incorporated into an entirety or region of the coating 40.

The magnetic components 52 may be a colloid, such as, but not limited to, a silicone base having a quantity of ferromagnetic material, or other magnetic material, mixed therein. In some examples, the ferromagnetic material may be carbonyl iron powder. Other ferromagnetic materials may include, but are not limited to, iron, cobalt nickel, rare-earth metals, and/or alloys or compounds thereof. It is contemplated that other types of magnetic materials may also be used, such as, but not limited to, ferrimagnetic, etc. The colloid may be combined using a standard mixing process. Carbonyl iron powder is available with filings of different lengths and densities which can be added to the silicone base to tailor the magnetic properties of the magnetic components 52. It is contemplated that the colloid coating may be applied via dip coating, spray coating, or can be manually applied in a sleeve form.

In one example an inner layer 40a or base layer (e.g., a silicone layer) may be formed over the struts 36. A colloid, including the ferromagnetic material, or other ferromagnetic component, may then be dropped or added in specific locations. The colloid may adhere to the base layer. An outer layer 40b may then be optionally applied over the inner layer 40a and the colloid to encapsulate the colloid, if desired or required. In such an instance, the inner and/or outer layers 40a, 40b may be dipped or sprayed as per instance. Alternatively, the inner and/or outer layers 40a, 40b and/or the colloid can be formed as sleeves and applied to the stent 10 in sleeve-form.

The variety of coating techniques may allow the magnetic components 52 to be applied to very specific areas of the stent 10 in any pattern desired to achieve a desired effect. It is further contemplated that the mechanical properties of the stent 10 may be tailored or customized by using coating materials having different or varying mechanical attributes (such as, but not limited to, various silicones) to achieve a desired effect. In one illustrative example, a silicone with higher elasticity and greater flexibility may be used for stents indicted for use in a more tortuous region of the body. This is just one example.

Generally, upon the application of a magnetic field, the magnetic components 52 may be moved radially outward or radially inward relative to the stent framework (e.g., struts 36). The movement of each magnetic component 52 may be localized within the cell spaces 46, if provided as discrete elements within the openings 46. For example, the magnetic components 52 may move radially inward and/or outward but may not move circumferentially and/or longitudinally.

The magnetic components 52 may be selectively exposed to a magnetic field to move the coating 40 of the stent 10. For example, a localized magnetic field may be generated by a bespoke or custom mobile console configured to be positioned exterior to the patient. The console may create an alternating magnetic field which generates an oscillating attraction and repulsion of the magnetic components 52 embedded in the coating 40 of the stent 10. As the magnetic field oscillates, the magnetic components 52 may move or oscillate thus causing the coating 40 to move or oscillate. This may promote the movement of bile, stones, food, or other bodily fluid or debris, through the stent 10 which may help prevent occlusive events.

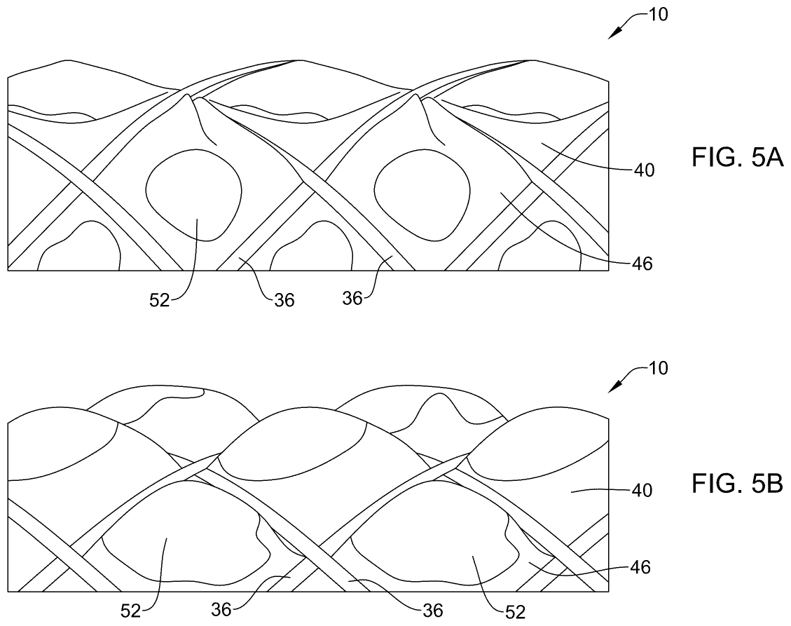

FIG. 5A illustrates a partial side view of an illustrative stent 10 including magnetic components 52 prior to magnetization and FIG. 5B illustrates a partial side view of the illustrative stent 10 of FIG. 5A including magnetic components 52 after magnetization. As can be seen in FIGS. 5A and 5B, the magnetic components 52 have been applied to specific areas of the stent 10. For example, the magnetic components 52 do not cover or extend over an entirety of the stent 10. Upon application of magnetization, localized movement of the magnetic components 52 may occur. For example, in the illustrated example, magnetization causes to the magnetic components 52 and the coating 40 to move radially outward from the plane of the struts 36 while maintaining each magnetic component 52 within a specific cell region 46. It is contemplated that in the absence of magnetization, the resiliency of the coating 40 may cause the coating 40 to return to the configuration in which the coating 40 is generally in the plane of the struts 36. Alternatively, or additionally, the resiliency of the coating 40 may cause the coating 40 to extend radially inward into the lumen 32 of the stent 10, at least temporarily. It is contemplated that if the struts 36 of the stent 10 are formed from or include a magnetic material, the struts 36 may move in response to the applied magnetic field. It is contemplated that struts 36 formed from a polymeric material may not be influenced by the applied magnetic field.

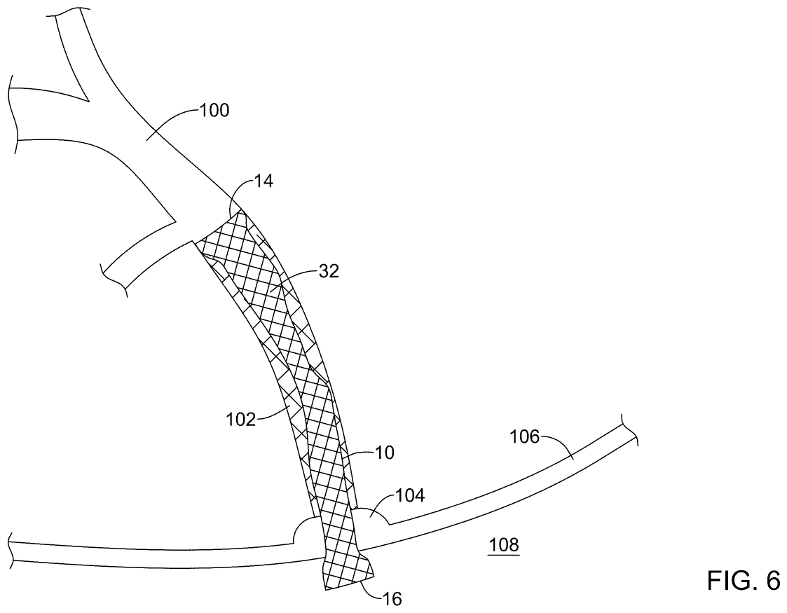

FIG. 6 depicts a schematic view of the illustrative stent 10 deployed within the biliary tree. In the illustrated example, the stent 10 is positioned within a biliary duct 100 and is positioned across a stricture 102. The distal end 16 of the stent 10 extends past the papillary mass 104, through the duodenal wall 106 and into the duodenum 108. The stent 10 may be positioned within the biliary tree via endoscopic retrograde cholangiopancreatography (ERCP). For example, the stent 10 may be guided to the biliary location and subsequently deployed, as shown in FIG. 6. The patient may be discharged with the stent 10 in-situ. For subsequent maintenance of the stent 10 (e.g., to prevent occlusion of the lumen 32 of the stent) after a period of time, the patient may be guided through a non-invasive treatment option. For example, the stent 10 may be periodically subjected to a magnetic field to cause the magnetic components 52, and the coating 40, to move and dislodge or prevent occlusions within the lumen 32 of the stent 10. It is contemplated that the time period between applications of the magnetic field may vary depending on whether or not the patient is symptomatic (e.g., showing signs of an occlusion) among other factors. In some cases, the time period between applications of the magnetic field may be in the range of minutes, hours, days, weeks, or months. It is further contemplated that the magnetic field may be applied at an outpatient facility, at a step-down facility, or as a home care option with the correct instructions. The application of a magnetic field to the stent 10 may be a less invasive and less costly intervention compared to surgical correction of an occlusion.

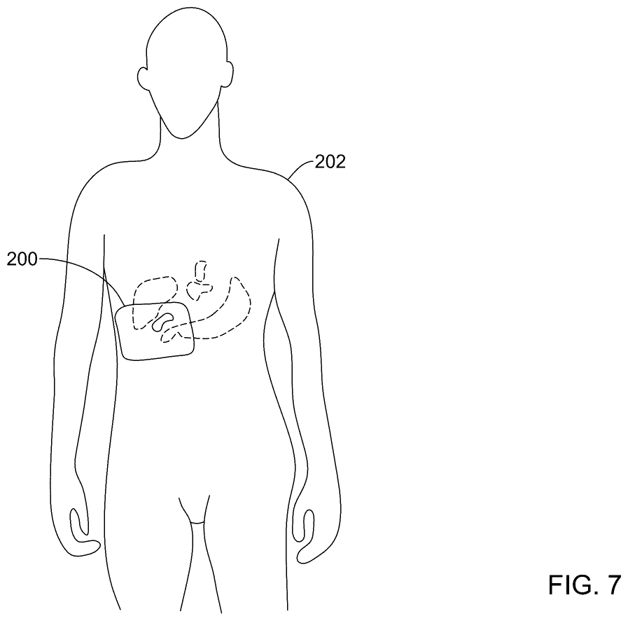

FIG. 7 illustrates a schematic view of the illustrative placement of a console 200 for generating a magnetic field relative to the body of the patient 202. The console 200 is positioned exterior to the patient 202 adjacent to the desired treatment region. In FIG. 7, the console 200 is positioned adjacent to the biliary tree. It is contemplated that the console 200 may be positioned at any location of the patient 202 to achieve the desired treatment, including, but not limited to the, the anterior side, the posterior side, the left side, or the right side, etc.

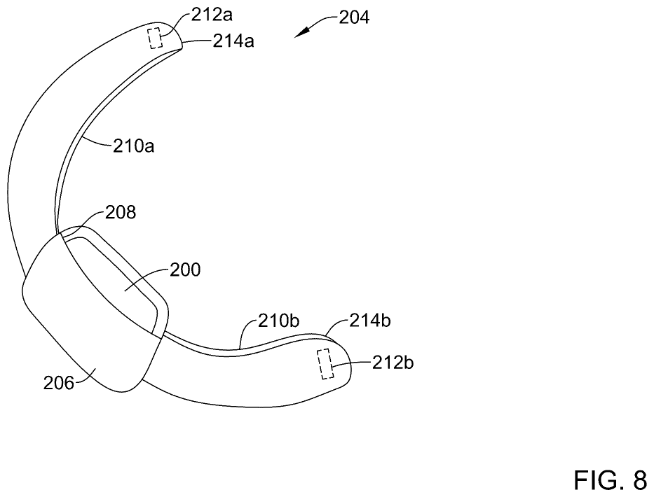

FIG. 8 illustrates a perspective view of an illustrative console system 204. The console system 204 may be portable and/or wearable such that the user may apply the magnetic field in a clinical setting, at home, or while going about their daily life. The console system 204 may include a body portion 206 defining a pocket or recess 208. The pocket 208 may be sized and shaped to receive the console 200 therein. One or more straps 210a, 210b may extend from the body portion 206. The one or more straps 210a, 210b may be configured to be secured about the body of the patient 202 to allow the patient 202 hands free operation of the console 200. The length of the straps 210a, 210b may be adjustable to accommodate different body locations and/or different body types. The one or more straps 210a, 210b may each include a fastening mechanism 212a, 212b adjacent to the free ends 214a, 214b thereof. The fastening mechanisms 212a, 212b may be releasably coupled to one another allow the straps 210a, 210b to be releasably secured about the patient 202. Some illustrative fastening mechanisms 212a, 212b may include, but are not limited to, hook and loop fasteners, snap mechanisms, buttons, zippers, etc. When activated, the console 200 generates a localized magnetic field which may attract and/repel the magnetic components 52 in the stent 10. In some embodiments, the console 200 may generate an alternating magnetic field which may generate an oscillating attraction and repulsion of the magnetic components 52 embedded in the coating 40 of the stent 10 in a cyclical manner thus causing the coating 40 to move radially outwards and inwards and encouraging sludge material to remain in motion. It is contemplated that the frequency of the oscillations may be adjusted to achieve the desired effect. In another example, a pulsed attractive-to-neutral cyclical field may be applied. In this instance, the magnetic components 52 may not necessarily be magnetically charged in their own aspect. Instead, the magnetic field may attract the magnetic components 52 in the coating 40 towards the applied source via the console 200 and when the source is removed the magnetic components 52 bounce back into position due to the elasticity of the coating 40, causing the magnetic components 52 and/or coating 40 to overshoot and protrude temporarily into the lumen 32 of the stent 10, thus causing a disruption of the coating 40 and encouraging sludge material to remain in motion.

The magnetic field oscillating frequency in either the alternating magnetic field or the pulsed magnetic field may be controlled and adjusted via the console 200. For example, the frequency may be increased for a more vigorous movement of the coating 40 of the stent 10 and the frequency may be decreased for a gentler movement of the coating 40 of the stent 10. In some cases, increasing the frequency may increase the radial distance by which the coating 40 of the stent 10 is displaced. As noted above, the movement or vibration of the coating 40 of the stent 10 may encourage internal material, such as, but not limited to, sludge, bile, stone debris, etc., to be keep moving through the lumen 32 of the stent 10 thus preventing or minimizing stagnation and occlusion. Additionally, the movement of the coating 40 may inhibit occlusive precursor materials such as biofilm from gaining adhesion to the surface of the stent 10. It is contemplated that if sludge, bile, stone debris, biofilm, etc., are left undisturbed they may cause early occlusion or other malfunctioning of the stent 10.

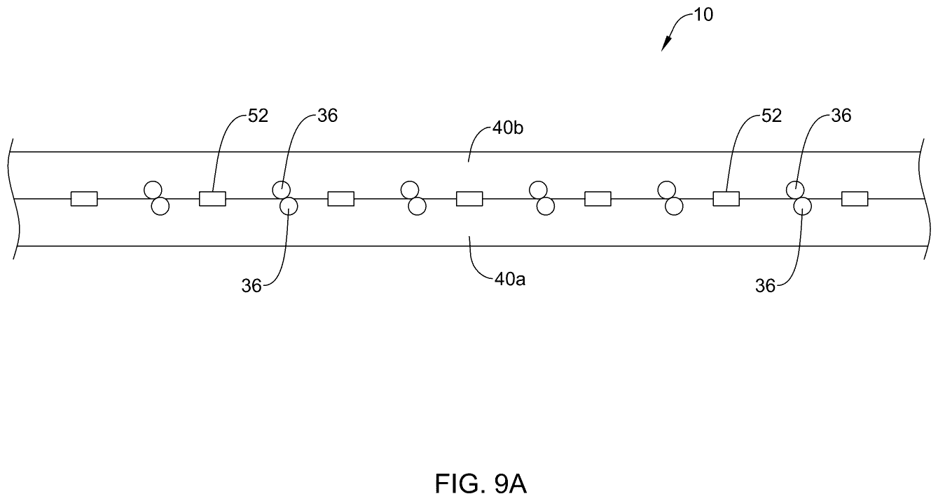

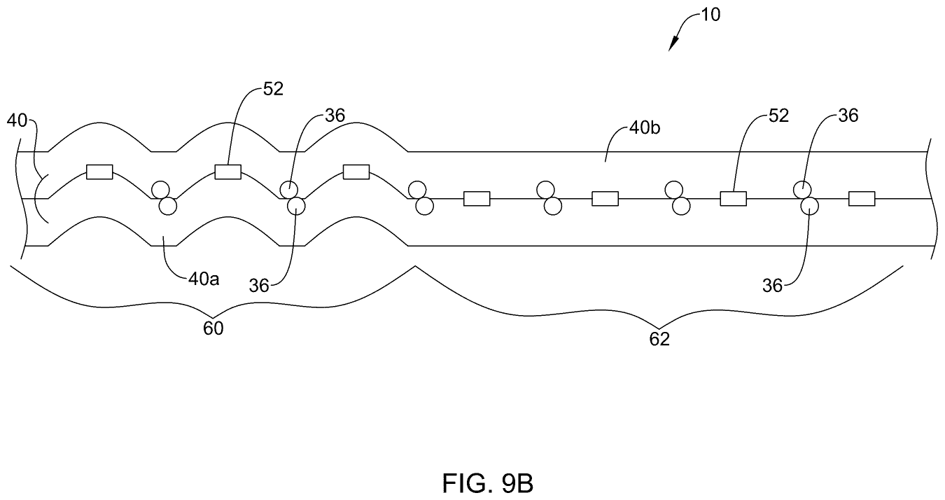

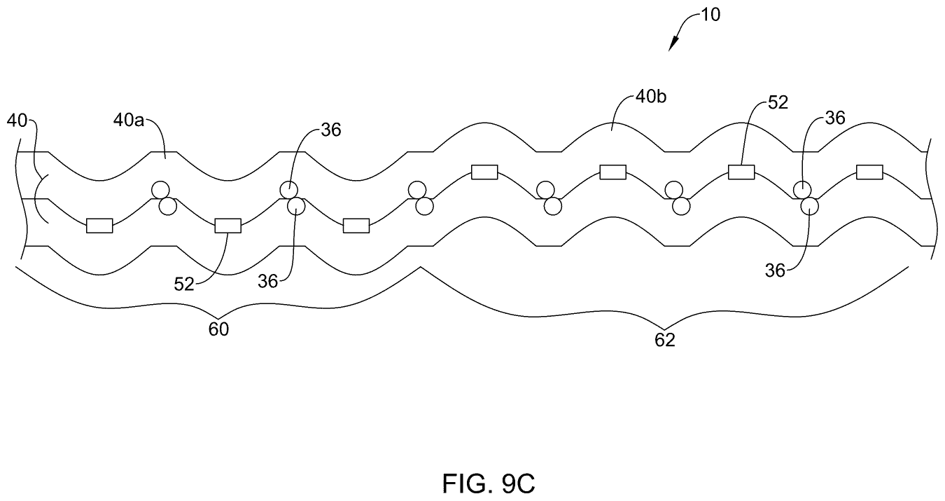

In some embodiments, the magnetic field may be selectively applied along the length of the stent 10. FIGS. 9A-9C illustrate a partial cross-sectional view of the stent 10 as a magnetic field is selectively applied. In FIG. 9A, the stent 10 is in an initial state with no magnetic field being applied. As can be seen, the inner layer of coating 40a, the outer layer of coating 40b, and the magnetic components 52 each lie within a same plane or generally parallel to a plane of the struts 36. In FIG. 9B, a magnetic field has been applied to a first region 60 of the stent 10 while there is zero or no magnetic field applied to a second region 62 of the stent 10. In the presence of the applied magnetic field, the magnetic components 52 in the first region 60 are drawn radially outward from the initial configuration, as shown in FIG. 9B while the magnetic components 52 in the second region 62 remain stationary. In FIG. 9C, the magnetic field has been removed from the first region 60 and applied to the second region 62. Once the magnetic field has been removed from the first region 60, the resiliency of the inner layer 40a and the outer layer 40b may cause the coating 40 and the magnetic components 52 to snap or move radially inwards within the lumen 32 of the stent 10, at least temporarily, as shown in FIG. 9C. Further, the magnetic components 52 in the second region 62 are drawn radially outward from the initial configuration. When the magnetic field is removed from the second region 62, the magnetic components 52 thereof may move radially inwards. The selective activation of the magnetic field over specific region of the stent 10 may allow the coating of the stent 10 to undulate in a wave-like pattern along a length thereof. It is further contemplated that an alternating magnetic field may be used to create a wave like movement along a length of the stent 10. However, this is not required. In some embodiments, an entirety of the stent 10 may be exposed to the magnetic field at a same time. In yet other embodiments, the magnetic field may be applied to a region of the stent 10 adjacent to the stricture 102 or other diseased site to pulse the stent 10 at the stricture 102 and thus manipulate the stricture.

As described above, the magnetic components 52 may be distributed about the length and/or circumference of the stent 10 in a number of different arrangements. In some examples, the magnetic components may be arranged to suit a particular condition under treatment or the perceived location of potential occlusion. For example, if an occlusion is predicted to potentially occur higher in the biliary duct (e.g., potentially due to stones, debris, etc.), the magnetic components 52 may be located closer to the proximal end 14 of the stent 10. In another example, if an occlusion is predicted to potentially occur at the lower aspect of the stent 10 (e.g., potentially due to food impaction, etc.), the magnetic components 52 may be located closer to the distal end 16 of the stent 10 (e.g., near the duodenal end).

It is further contemplated that removal of a stent 10 after a period of indwelling may be difficult. For example, an adhesive build-up between the outer coating 40b of the stent 10 and the lumen wall and/or between residual bile liquids and the surrounding lumen wall may inhibit removal of the stent 10. The adhesive build-up may be exacerbated by the radial force of the struts 36 of the stent 10 and/or the presence of holes in the coating 40 which allow for tissue ingrowth. It is contemplated that routine exposure to the controlled magnetic field generated by the magnetic source 200 can cause the coating 40 including the magnetic components 52 to vibrate, move, oscillate, etc. as described herein to influence and encourage the temporary separation of the stent 10 from the surrounding lumen and the breaking of the temporary adhesive bond. This may leave the stent 10 with a weaker bond and allow for easier removal. Repeated exposure to the magnetic field at predetermined intervals may prevent or minimize long-term embedding of the stent 10 with the lumen wall and improve removal potential. Similarly, localized movement of the coating 40 and the magnetic components 52 may cause the tissue ingrowth to be separated from any holes in the coating 40 and allow for easier stent removal. In this manner, it is envisaged that after a period of indwell, when a stent 10 is scheduled for removal, a program of magnetic exposure prior to intervention may cause easier stent extraction. In yet other examples, the magnetic field may be strategically applied to perform remote extraction of the stent 10 with the stent 10 passing through the natural vessel, without inserting additional medical devices into the body.

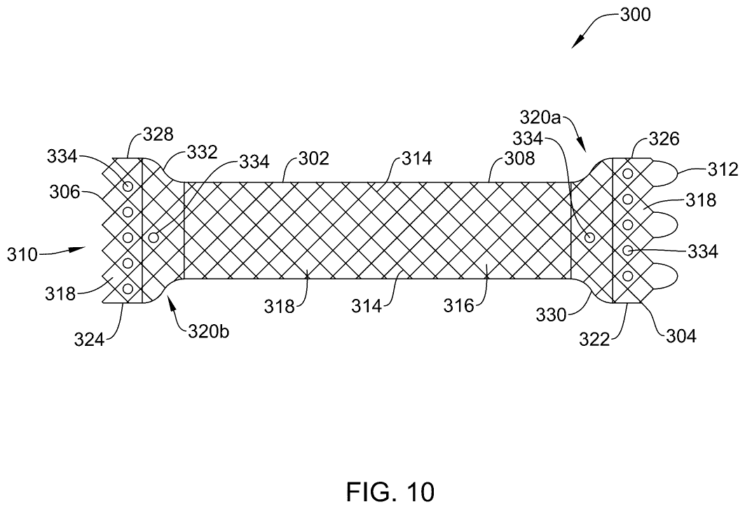

Further, the inclusion of a coating 40 including magnetic components 52 with a partially covered (PC) stent, may allow for efficient extraction of the stent 10 even given that the partially coated stent is designed for tissue ingrowth. Typically, a partially covered stent, if requiring removal may require prior argon plasma coagulation (APC), an overtube technique (OT) and/or a stent-in-stent (SIS) technique which may all add to total procedural cost and/or complexity. FIG. 10 illustrates a side view of another illustrative endoluminal implant 300, such as, but not limited to, a stent. The stent 300 may be similar in form and function to the stent 10 described herein. In some instances, the stent 300 may be formed from an elongated tubular member 302. While the stent 300 is described as generally tubular, it is contemplated that the stent 300 may take any cross-sectional shape desired. The stent 300 may have a first, or proximal end 304, a second, or distal end 306, and an intermediate region 308 disposed between the first end 304 and the second end 306. The stent 300 may include a lumen 310 extending from a first opening adjacent the first end 304 to a second opening adjacent to the second end 306 to allow for the passage of food, fluids, etc.

The stent 300 may be expandable from a first radially collapsed configuration (not explicitly shown) to a second radially expanded configuration. In some cases, the stent 300 may be deployed to a configuration between the collapsed configuration and a fully expanded configuration. The stent 300 may be structured to extend across a stricture and to apply a radially outward pressure to the stricture in a lumen to open the lumen and allow for the passage of substances.

In some embodiments, the proximal end 304 of the stent 300 may include a plurality of loops 312. The loops 312 may be configured to receive a retrieval tether or suture (not explicitly shown) interwoven therethrough, or otherwise passing through one or more of the loops 312. The retrieval suture may be used to collapse and retrieve the stent 300, if so desired. For example, the retrieval suture may be pulled like a drawstring to radially collapse the proximal end 304 of the stent 300 to facilitate removal of the stent 300 from a body lumen.

The stent 300 may have a woven structure, fabricated from a number of filaments or struts 314 forming a tubular wall. In some embodiments, the stent 300 may be knitted or braided with a single filament or strut interwoven with itself and defining open cells 316 extending through the thickness of the tubular wall of the stent 300. In other embodiments, the stent 300 may be braided with several filaments or struts interwoven together and defining open cells 316 extending along a length and around the circumference of the tubular wall of the stent 300. The open cells 316 may each define an opening from an outer surface of the tubular wall to an inner surface of the tubular wall (e.g., through a thickness thereof) that is free from the filaments or struts 314. In another embodiment, the stent 300 may be knitted. In yet another embodiment, the stent 300 may be of a knotted type. In still another embodiment, the stent 300 may be a laser cut tubular member. A laser cut tubular member may have an open and/or closed cell geometry including one or more interconnected monolithic filaments or struts defining open cells 316 therebetween, with the open cells 316 extending along a length and around the circumference of the tubular wall. The open cells 316 may each define an opening from an outer surface of the tubular wall to an inner surface of the tubular wall (e.g., through a thickness thereof) that is free from the interconnected monolithic filaments or struts. In some instances, an inner and/or outer surface of the tubular wall of the stent 300 may be partially, covered with a polymeric covering or coating 318. The covering or coating 318 may extend across and/or occlude one or more, or a plurality of the cells 316 defined by the struts or filaments 314. The coating 318 may include more than one layer and may be similar in form and function to the coating 40 described herein. In some cases, the stent 300 may be a self-expanding stent (SES), although this is not required.

The coating 318 may partially cover the open cells 316 while leaving some cells 316 or circumferential regions 320a, 320b free from the coating 318. The regions 320a, 320b free from the coating 318 may allow for tissue in-growth in selective areas of the stent 300. In the illustrated embodiment, the stent 300 includes two regions 320a, 320b generally or mostly free from the coating 318. A first region 320a may be proximate to and longitudinally spaced from the proximal end 304 and the second region 320b may be proximate to and longitudinally spaced from the distal end 306. This is just one example. It is contemplated that the mostly coating-free regions 320a, 320b may start at or extend to the proximal end 304 or distal end 306, respectively. Other configurations of the mostly coating-free regions 320a, 320b may be used as desired. In some examples, the stent 300 may include only a single coating-free region or may include more than two coating-free regions.

In some instances, in the radially expanded configuration, the stent 300 may include a first end region 322 proximate the proximal end 304 and a second end region 324 proximate the second end 306. In some embodiments, the first end region 322 and the second end region 324 may include retention features or anti-migration flared regions 326, 328 having enlarged diameters relative to the intermediate portion 308. The anti-migration flared regions 326, 328, which may be positioned adjacent to the first end 304 and the second end 306 of the stent 300, may be configured to engage an interior portion of the walls of the body lumen. In some embodiments, the retention features, or flared regions 326, 328 may have a larger diameter than the cylindrical intermediate region 308 of the stent 300 to prevent the stent 300 from migrating once placed in the body lumen. It is contemplated that the transition 330, 332 from the cross-sectional area of the intermediate region 308 to the retention features or flared regions 326, 328 may be gradual, sloped, or occur in an abrupt step-wise manner, as desired.

In some embodiments, the first anti-migration flared region 326 may have a first outer diameter and the second anti-migration flared region 328 may have a second outer diameter. In some instances, the first and second outer diameters may be approximately the same, while in other instances, the first and second outer diameters may be different. In some embodiments, the stent 300 may include only one or none of the anti-migration flared regions 326, 328. For example, the first end region 322 may include an anti-migration flare 326 while the second end region 324 may have an outer diameter similar to the intermediate region 308. It is further contemplated that the second end region 324 may include an anti-migration flare 328 while the first end region 322 may have an outer diameter similar to an outer diameter of the intermediate region 308. In some embodiments, the stent 300 may have a uniform outer diameter from the first end 304 to the second end 306. It is contemplated that the outer diameter of the stent 300 may be varied to suit the desired application.

It is contemplated that the elongated tubular member of the stent 300 can be made from a number of different materials such as, but not limited to, metals, metal alloys, shape memory alloys, and/or polymers, as desired, enabling the stent 300 to be expanded into shape when accurately positioned within the body. In some instances, the material may be selected to enable the stent 300 to be removed with relative ease as well. For example, the elongated tubular member of the stent 300 can be formed from alloys such as, but not limited to, nitinol and Elgiloy®. Depending on the material selected for construction, the stent 300 may be self-expanding or require an external force to expand the stent 300. In some embodiments, composite filaments may be used to make the stent 300, which may include, for example, an outer shell or cladding made of nitinol and a core formed of platinum or other radiopaque material. It is further contemplated the elongated tubular member of the stent 300 may be formed from polymers including, but not limited to, polyethylene terephthalate (PET). In some instances, the filaments of the stent 300, or portions thereof, may be bioabsorbable or biodegradable, while in other instances the filaments of the stent 300, or portions thereof, may be biostable.

The stent 300 may include one or more magnetic components 334. The one or more magnetic components 334 may be similar in form and function the magnetic components 52 described herein. For example, the magnetic components 334 may be encapsulated between an inner layer and an outer layer of the coating 318. However, this is not required. In some cases, the magnetic component 334 may be on an inner surface of the inner layer or an outer surface of the outer layer of the coating 318. The one or more magnetic components 334 may be a colloid, such as, but not limited to, a silicone base having a quantity of ferromagnetic material, or other magnetic material, mixed therein.

The magnetic components 334 may be positioned adjacent to or may even extend into the mostly coating-free regions 320a, 320b. The magnetic components 334 may be arranged in any uniform or non-uniform arrangement, as desired. In some cases, the magnetic components 334 may be discrete elements disposed within the cells 316. In other cases, the magnetic components 334 may be elongated bars or strips. In yet other cases, the magnetic components 334 may substantially cover a portion of the stent 300. In some embodiments, “islands” or regions of coating 318 including one or more magnetic components 334 may extend into the generally coating-free regions 320a, 320b. This may allow for areas of tissue in-growth while also allowing the movement of the magnetic components 334 in response to an applied magnetic field to move the mostly coating-free regions 320a, 320b which may encourage tissue dislodgement.

The stents, delivery systems, and the various components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-clastic nitinol; other nickel alloys such as nickel-chromium-molybdenum alloys, nickel-copper alloys, nickel-cobalt-chromium-molybdenum alloys, nickel-molybdenum alloys, other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys; platinum enriched stainless steel; titanium; combinations thereof; and the like; or any other suitable material.

Some examples of suitable polymers for the stents or delivery systems may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like.

In at least some embodiments, portions or all of the stents or delivery systems may also be doped with, made of, or otherwise include a radiopaque material. Radiopaque materials are generally understood to be materials which are opaque to RF energy in the wavelength range spanning x-ray to gamma-ray (at thicknesses of <0.005″). These materials are capable of producing a relatively dark image on a fluoroscopy screen relative to the light image that non-radiopaque materials such as tissue produce. This relatively bright image aids the user of the stents or delivery systems in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the stents or delivery systems to achieve the same result.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A stent, the stent comprising:
an elongated tubular member comprising at least one strut forming a tubular wall having a plurality of cells extending through a thickness of the tubular wall, the elongated tubular member configured to move between a radially collapsed configuration and a radially expanded configuration;
a coating disposed on the elongated tubular member and spanning at least some of the plurality of cells; and
one or more magnetic components disposed on or within the coating.

2. The stent of claim 1, wherein the coating comprises an inner layer and an outer layer.

3. The stent of claim 2, wherein the one or more magnetic components are disposed between the inner layer and the outer layer of the coating.

4. The stent of claim 1, wherein the one or more magnetic components are discrete elements disposed within at least some of the plurality of cells.

5. The stent of claim 1, wherein the one or more magnetic components are elongate strips extending over the at least one strut.

6. The stent of claim 1, wherein the one or more magnetic components are spaced along a length of the elongated tubular member.

7. The stent of claim 1, wherein the one or more magnetic components are spaced about a circumference of the elongated tubular member.

8. The stent of claim 1, wherein the coating forms a pocket within at least some of the plurality of cells.

9. The stent of claim 1, wherein the coating extends from a proximal end to a distal end of the elongated tubular member.

10. The stent of claim 1, wherein the coating covers less than an entirety of the elongated tubular member.

11. The stent of claim 10, wherein at least one of the one or more magnetic components are positioned adjacent to a coating-free region of the elongated tubular member.

12. The stent of claim 1, wherein the one or more magnetic components comprise a silicone base and a magnetic material.

13. The stent of claim 12, wherein the magnetic material comprises carbonyl iron.

14. The stent of claim 1, wherein in response to an applied magnetic field, the one or more magnetic components are configured to move radially inward and/or radially outward.

15. The stent of claim 14, wherein the applied magnetic field is a pulsed magnetic field or an alternating magnetic field.

16. A stent, the stent comprising:
an elongated tubular member comprising at least one strut forming a tubular wall having a plurality of cells extending through a thickness of the tubular wall, the elongated tubular member configured to move between a radially collapsed configuration and a radially expanded configuration;
a coating disposed on the elongated tubular member and spanning at least some of the plurality of cells; and
a plurality of magnetic components embedded within the coating, the plurality of magnetic components including a silicone base and carbonyl iron;
wherein in response to an applied magnetic field, the one or more magnetic components are configured to move radially inward and/or radially outward relative to the at least one strut.

17. A method for preventing occlusion of a stent, the method comprising:
delivering a stent to a target location within a body, the stent comprising:
an elongated tubular member comprising at least one strut forming a tubular wall having a plurality of cells extending through a thickness of the tubular wall, the elongated tubular member configured to move between a radially collapsed configuration and a radially expanded configuration;
a coating disposed on the elongated tubular member and spanning at least some of the plurality of cells; and
one or more magnetic components disposed on or within the coating;
positioning a console configured to supply a magnetic field exterior to the body and adjacent to the target location; and
periodically activating the magnetic field;
wherein in response to the magnetic field being applied, the one or more magnetic components are configured to move radially inward and/or radially outward relative to the at least one strut.

18. The method of claim 17, wherein the magnetic field is a pulsed magnetic field.

19. The method of claim 17, wherein the magnetic field is an alternating magnetic field.

20. The method of claim 17, wherein the console is configured to be releasably secured about the body.
